(19) United States
(12) Patent Application Publication
(10) Pub. No.: US 2020/0345303 A1
(43) Pub. Date: Nov. 5, 2020

(54) BELT, BLOOD PRESSURE MEASURING DEVICE, AND METHOD OF MANUFACTURING BELT

(71) Applicants: OMRON CORPORATION, Kyoto-shi (JP); OMRON HEALTHCARE CO., LTD., Muko-shi (JP)

(72) Inventors: Noboru KOHARA, Okayama-shi (JP); Tomoyuki NISHIDA, Kyoto-shi (JP); Shinji MIZUNO, Yasu-shi (JP); Hirokazu TANAKA, Otsu-shi (JP); Keitaro NAGANO, Kyoto (JP)

(73) Assignees: OMRON CORPORATION, Kyoto-shi (JP); OMRON HEALTHCARE CO., LTD., Muko-shi (JP)

(21) Appl. No.: 16/929,848

(22) Filed: Jul. 15, 2020

Related U.S. Application Data
(63) Continuation of application No. PCT/JP2019/000347, filed on Jan. 9, 2019.

(30) Foreign Application Priority Data
Jan. 15, 2018 (JP) 2018-004049

(51) Int. Cl. A61B 5/00; A61B 5/022; B29B 11/08; B29C 53/02

(52) U.S. Cl. CPC A61B 5/6831; A61B 5/02233; B29B 11/08; A61B 5/0235; A61B 2562/0247; A61B 2562/168; B29C 53/02

(57) ABSTRACT

A belt includes; a first belt portion including a cover layer formed of a resin material and a first insert layer disposed within the cover layer and formed of a high-tensile material having a tensile strength higher than that of the resin material, the first belt portion having a bent shape along a circumferential direction of a living body; and a second belt portion configured to be connected to the first belt portion.

### CROSS-REFERENCE TO RELATED APPLICATIONS

This is a Continuation Application of PCT application No. PCT/JP2019/000347, filed Jan. 9 2019, which was published under PCT Article 21(2) in Japanese.

This application is based upon and claims the benefit of priority from Japanese Patent Application No. 2018-004049, filed Jan. 15, 2018, the entire contents of which are incorporated herein by reference.

### BACKGROUND

### Field

The present invention relates to a belt, a blood pressure measuring device, and a method of manufacturing the belt.

### Description of the Related Art

In recent years, a blood pressure measuring device for the use of blood pressure measurement has been utilized not only in medical facilities but also in households as a means to confirm health condition.

For example, a blood pressure measuring device includes a device main body, a belt, a curler, a cuff structure, and a fluid circuit.

The device main body, for example, forms a flow path of a fluid, and includes a pump that supplies the fluid and a pressure sensor that detects pressure. The belt includes, for example, a first belt extending to one side of the device main body and a second belt extending to the other side of the device main body. The first belt is formed of a band-shaped member made of a resin material, for example, and includes a buckle at an end of the first belt. The second belt is made of a resin material in a band shape, for example, and has a hole that engages with the buckle of the first belt. The curler is made of a resin material, for example, and has a predetermined bent shape. The cuff structure includes a pressing cuff and a sensing cuff formed in a bag shape, and is wound along a living body. The internal space of the cuff structure is connected to the flow path of the device main body.

### SUMMARY

A blood pressure measuring device measures blood pressure by detecting vibrations of an arterial wall, by, for example, inflating and deflating a cuff with a belt, a curler, and the cuff wound around an upper arm, a wrist, or the like of a living body, and detecting the pressure of the cuff with the use of a pressure sensor provided to a device main body (e.g., Jpn. Pat. Appln. KOKAI Publication No 2017-121479).

In general, in a blood pressure device, a force that pulls a belt in a circumferential direction of a living body such as a wrist acts when a cuff structure is inflated. When the belt is stretched in the circumferential direction, the measurement accuracy of blood pressure is affected.

According to one aspect, there is provided a belt including: a first belt portion including a cover layer formed of a resin material and a first insert layer disposed within the cover layer and formed of a high-tensile material having a tensile strength higher than that of the resin material, the first belt portion having a bent shape along a circumferential direction of a living body; and a second belt portion configured to be connected to the first belt portion.

According to this aspect, since the high-tensile material is disposed in the cover layer, it is possible to reduce the likelihood of stretching of the belt when an external force in the circumferential direction is applied. Since the belt is formed in a bent shape, it demonstrates high workability when the belt is wound around and attached to the living body.

Herein, the high-tensile material is, for example, a member made of a material having a high tensile strength. The high-tensile material is, for example, a resin material, and examples thereof include high-strength polyarylate fibers, liquid crystal polymers, PET resins, and PEN resins.

In the belt according to the above aspect, the first belt portion further includes a second insert layer disposed within the cover layer and formed of a thermoplastic resin; the cover layer is formed of a thermosetting resin; and a tensile strength of the high-tensile material in the circumferential direction of the living body is higher than that of the thermosetting resin forming the cover layer.

According to this aspect, the cover layer and the insert layer are heated at the same temperature so as to make the properties of the cover layer and the insert layer different from each other, whereby the belt can be easily bent.

In the belt according to the above aspect, the high-tensile material includes at least one of a high-strength polyarylate fiber (Vectran fiber (registered trademark)), a liquid crystal polymer, a PET resin, and a PEN resin.

According to this aspect, since at least one of the high-strength polyarylate fiber, the liquid crystal polymer, the PET resin, and the PEN resin is included, it is possible to obtain a lightweight and thin belt while securing its tensile strength, as compared to a case where the entire belt is formed of the material of the cover layer.

In the belt according to the above aspect, the high-tensile material is formed in a mesh shape.

According to this aspect, since it is possible to improve the bonding between the cover layer and the high-tensile material by covering the mesh-shaped high-tensile material with the cover layer, it is possible to make the high-tensile material and the resin layer less likely to be peeled off.

According to one aspect, there is provided a blood pressure measuring device including: the belt; a curler disposed on a side of the belt closer to a living body and having a bent shape along the living body; a bag-shaped cuff disposed on one side of the curler, configured to be wound around the living body, and further configured to be inflated when a fluid is supplied to an inner space of the cuff; and a supply device configured to be attached to the belt, also configured to form a flow path connected to the inner space of the cuff, and further configured to supply the fluid to the cuff.

Herein, the fluid includes liquid and air. The cuff is wound around an upper arm, a wrist, or the like of a living body when blood pressure is measured, and is inflated by supplying a fluid thereto. Examples of the cuff include a pressing cuff provided in a blood pressure measuring device that measures blood pressure at the wrist, a sensing cuff, and a cuff provided in a blood pressure measuring device that measures blood pressure at the upper arm. The cuff herein may be a bag-shaped structure such as an air bag constituting a pressing cuff. Also herein, the supply device is a device main body of a blood pressure measuring device including a pump and a flow path.

According to this aspect, the belt provided in the blood pressure measuring device can suppress elongation of the belt even when a stress pulling the belt is applied in the circumferential direction of the living body due to the inflation of the cuff. Therefore, high measurement accuracy can be maintained.

According to one aspect, there is provided a method of manufacturing a belt, the method including: a preform forming step of forming a band-shaped preform by insert molding, the preform comprising a first insert material disposed inside a cover layer formed of a thermosetting resin, the first insert material made of a high-tensile material having a tensile strength higher than that of the thermosetting resin; and a bending step of bending the preform.

According to this aspect, the bending into a desired shape can be easily performed after performing molding to form a simple shape. Therefore, the molding process and the configuration of the mold can be simplified.

In the method of manufacturing a belt according to the above aspect, the preform forming step includes: disposing a thermosetting resin material forming the cover layer around the first insert material and a second insert material that is made of a thermoplastic resin; curing the thermosetting resin and softening the second insert material by heating; thereby performing insert molding of the preform, and the bending step includes a curing step of bending the preform and curing the second insert material at a temperature lower than a temperature at which the insert molding is performed.

According to this aspect, the bending into a desired shape can be easily performed by adjusting the temperature at which insert molding is performed and the temperature at which cooling is performed, the bending process becomes easy.

The present invention can provide a belt, a blood pressure measuring device, and a method of manufacturing a belt that can improve the measurement accuracy of blood pressure.

### BRIEF DESCRIPTION OF THE DRAWINGS

FIG. 1 is a perspective view of a configuration of a blood pressure measuring device according to a first embodiment of the present invention.

FIG. 2 is a perspective view of a configuration of the blood pressure measuring device.

FIG. 3 is an exploded view of a configuration of the blood pressure measuring device.

FIG. 4 is a block diagram showing a configuration of the blood pressure measuring device.

FIG. 5 is a perspective view of a configuration of a first belt of the blood pressure measuring device.

FIG. 6 is a cross-sectional view of a configuration of the first belt.

FIG. 7 is an explanatory diagram showing a method of manufacturing the belt of the blood pressure measuring device.

FIG. 8 is a perspective view of a configuration of a preform of the belt of the blood pressure measuring device.

FIG. 9 is a cross-sectional view of a configuration of a second belt of the blood pressure measuring device.

FIG. 10 is a perspective view of another configuration of the blood pressure measuring device.

FIG. 11 is a perspective view of a configuration of a device main body of the blood pressure measuring device.

FIG. 12 is a plan view of an internal configuration of the device main body.

FIG. 13 is a plan view of an internal configuration of the device main body.

FIG. 14 is a plan view of a configuration of a cuff structure of the blood pressure measuring device.

FIG. 15 is a cross-sectional view of configurations of a curler and the cuff structure of the blood pressure measuring device.

FIG. 16 is a cross-sectional view of configurations of the curler and the cuff structure.

FIG. 17 is an explanatory diagram schematically showing a configuration of the cuff structure in which a pressing cuff is inflated.

FIG. 18 is a cross-sectional diagram schematically showing a configuration of the cuff structure in which a pressing cuff is inflated.

FIG. 19 is a flowchart showing an example of the use of the blood pressure measuring device.

FIG. 20 is a perspective diagram showing an example in which the blood pressure measuring device is worn on a wrist.

FIG. 21 is a perspective diagram showing an example in which the blood pressure measuring device is worn on a wrist.

FIG. 22 is a perspective diagram showing an example in which the blood pressure measuring device is worn on a wrist.

### DETAILED DESCRIPTION

### First Embodiment

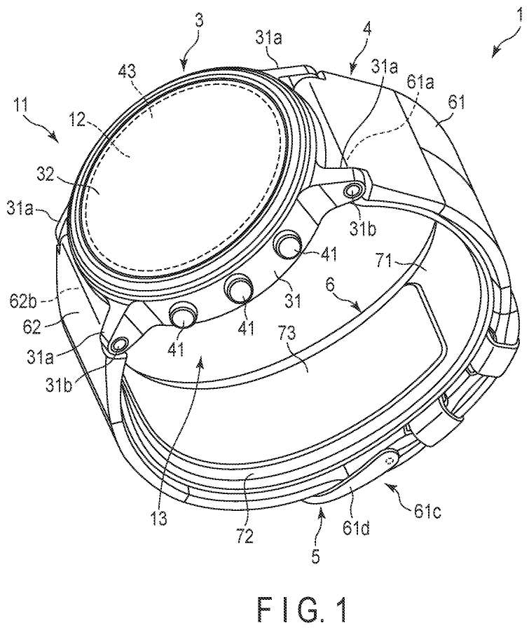

Hereinafter, an example of a blood pressure measuring device 1 according to a first embodiment of the present invention will be described with reference to FIGS. 1 to 18.

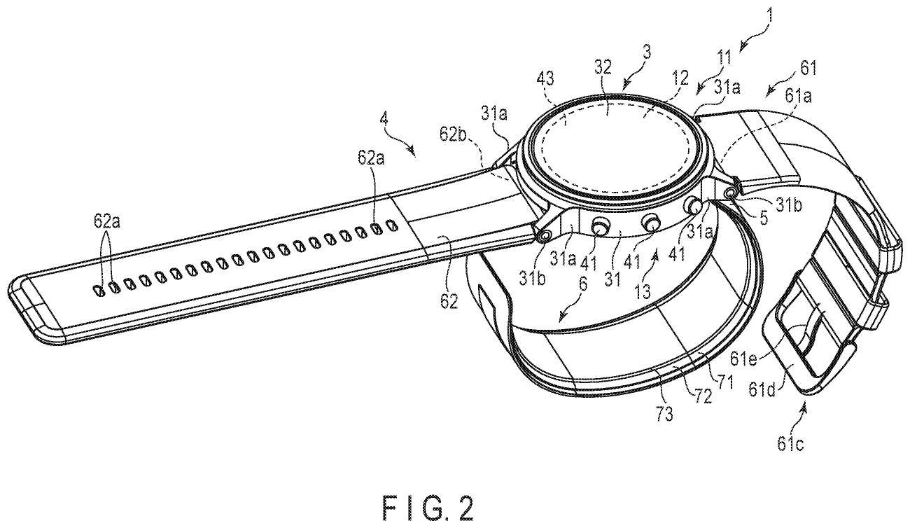

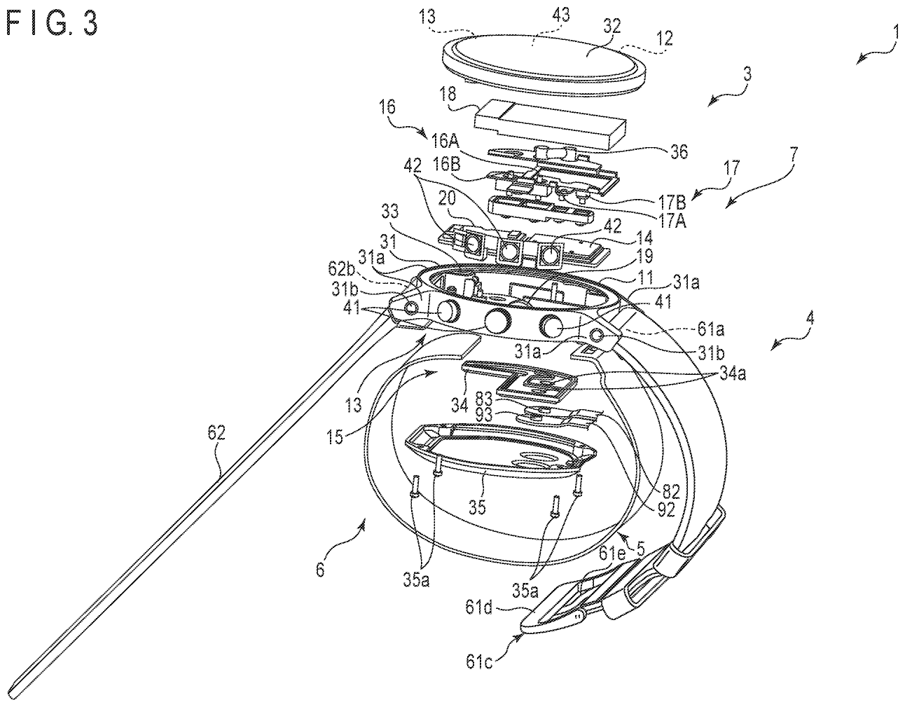

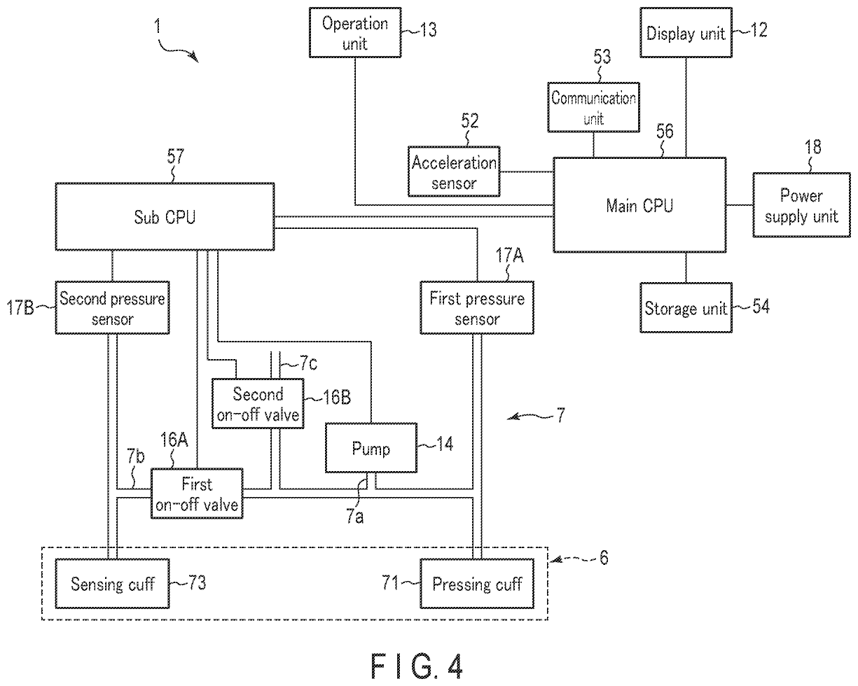

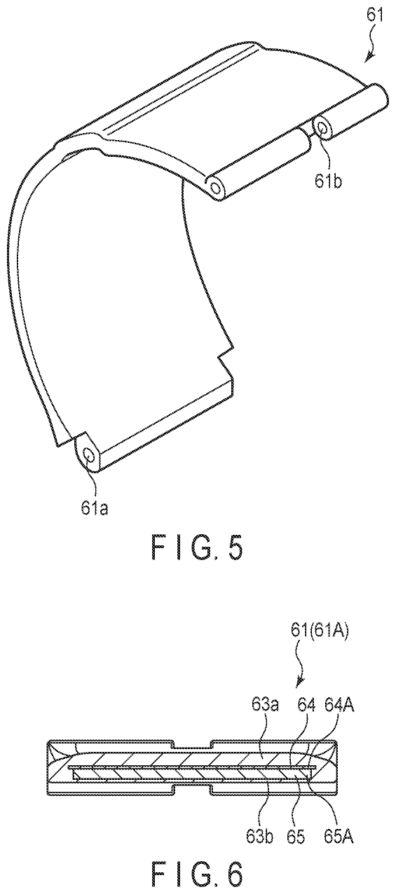

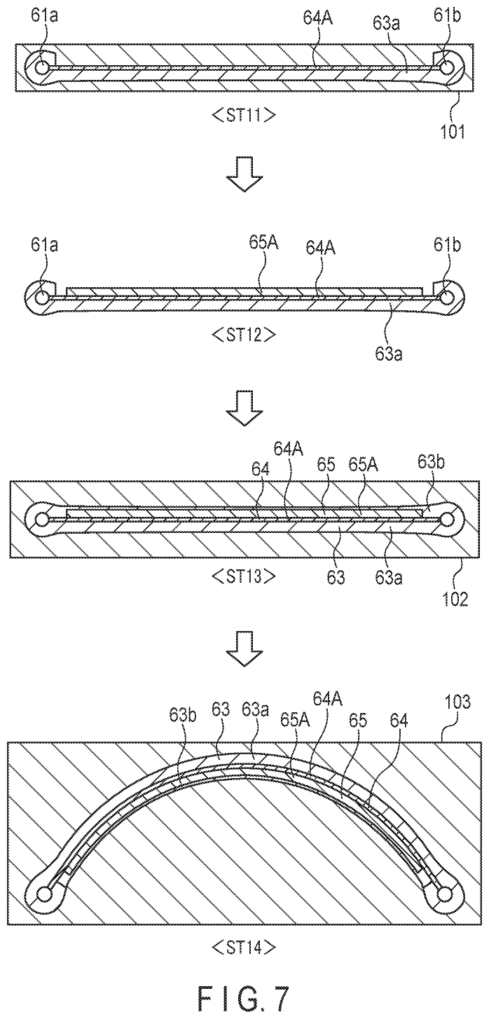

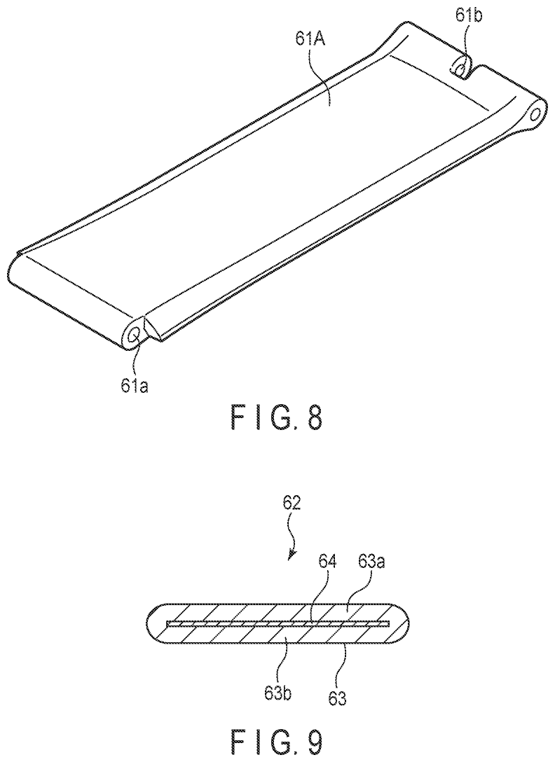

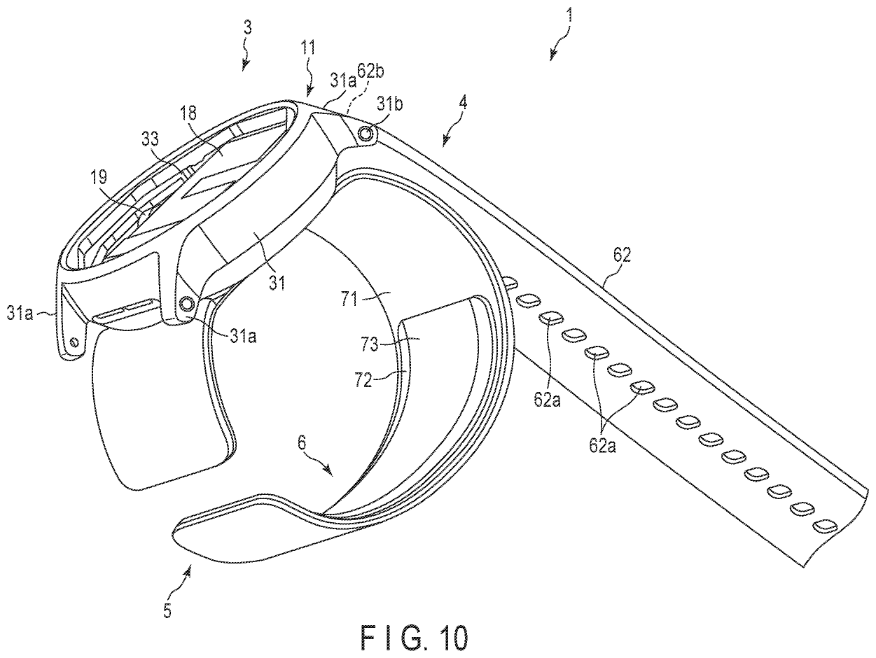

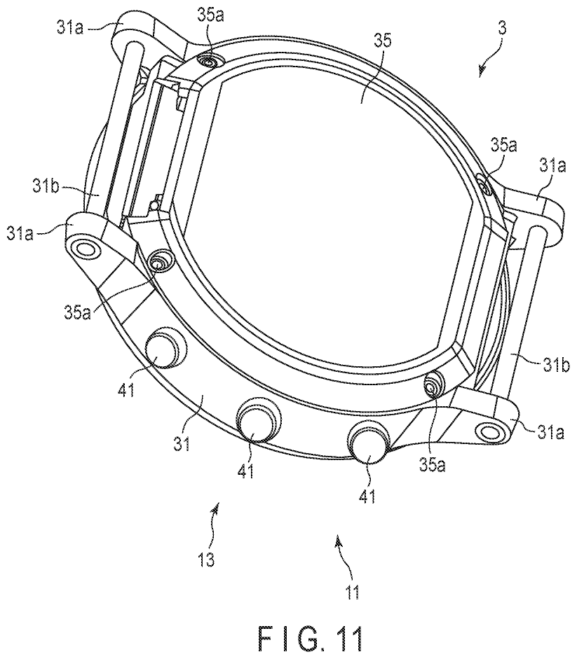

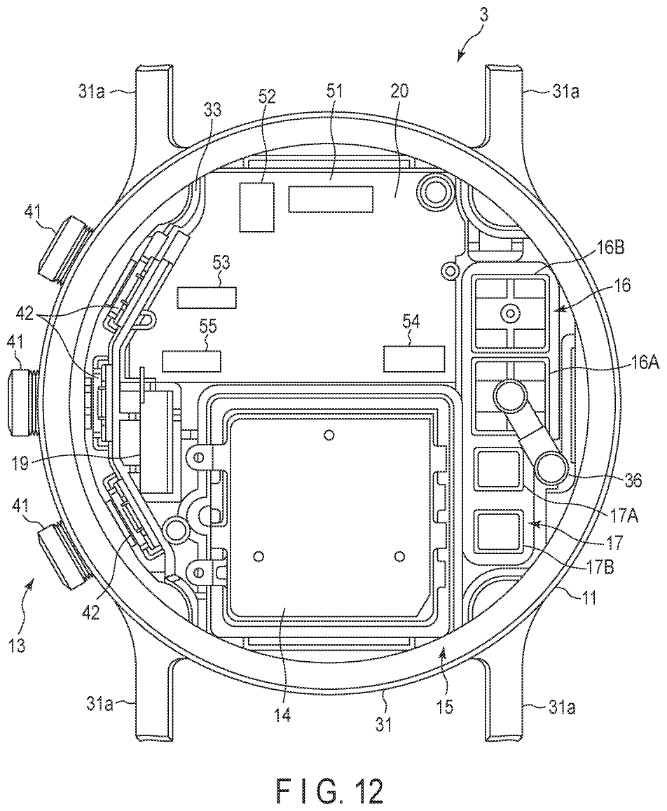

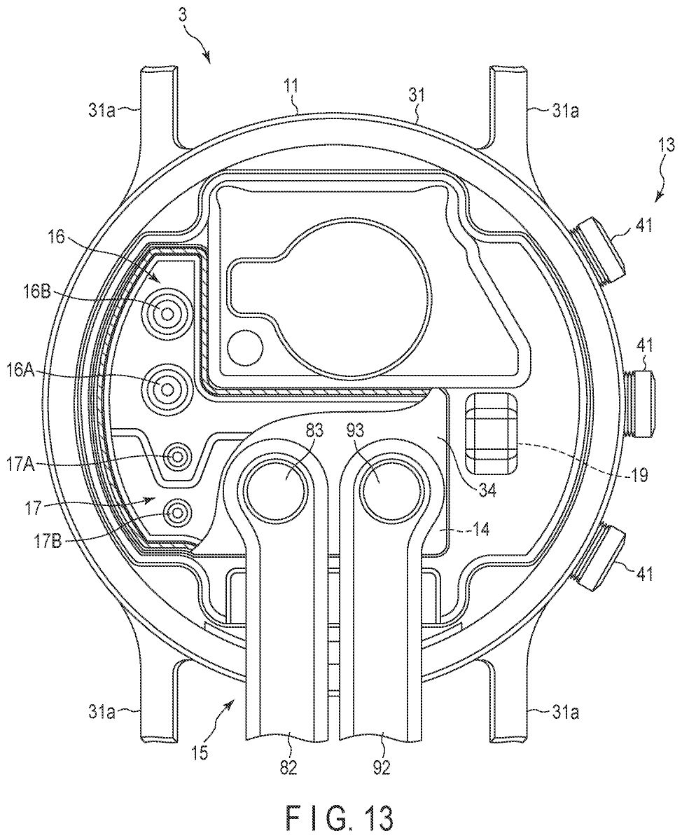

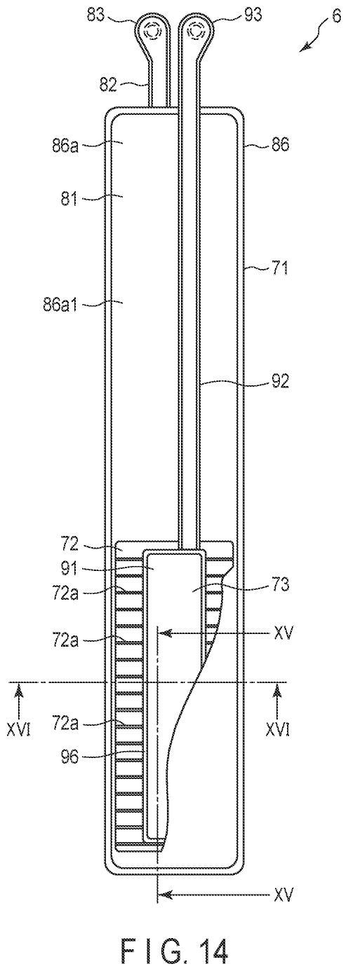

FIG. 1 is a perspective view of a configuration of the blood pressure measuring device 1 according to the first embodiment of the present invention with a belt 4 closed. FIG. 2 is a perspective view of a configuration of the blood pressure measuring device 1 with the belt 4 opened. FIG. 3 is an exploded view of a configuration of the blood pressure measuring device 1. FIG. 4 is a block diagram showing a configuration of the blood pressure measuring device 1. FIGS. 5 and 6 are a perspective view and a cross-sectional view of a configuration of a first belt portion 61. FIG. 7 is an explanatory diagram showing a method of manufacturing the first belt portion 61. FIG. 8 is a perspective view of a configuration of a preform 61A. FIG. 9 is a cross-sectional view of a configuration of a second belt portion 62. FIG. 10 is a perspective view of another configuration of the blood pressure measuring device 1. FIG. 11 is a perspective view of a configuration of a device main body 3 of the blood pressure measuring device 1, as viewed from a back cover 35 side. FIGS. 12 and 13 are plan views of an internal configuration of the device main body 3, as viewed from a windshield 32 side and the back cover 35 side, respectively. FIG. 14 is a plan view of a configuration of a cuff structure 6 of the blood pressure measuring device 1, as viewed from a sensing cuff 73 side.

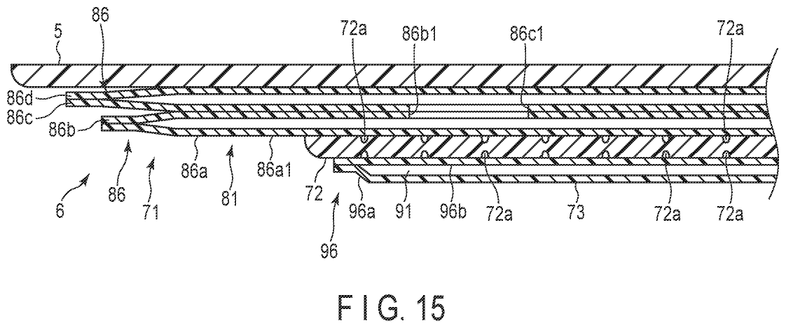

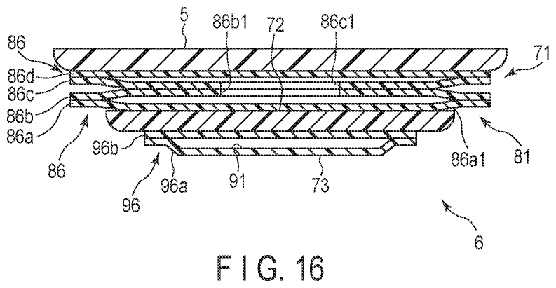

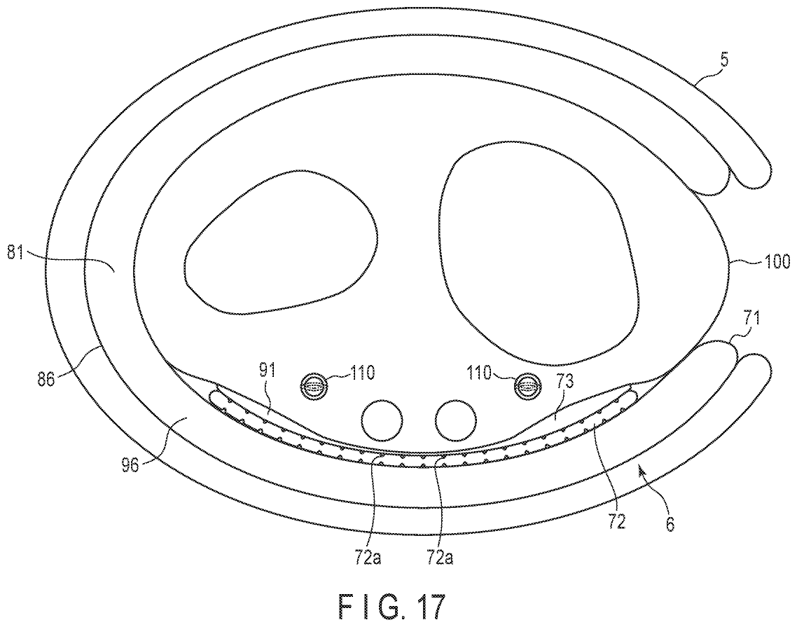

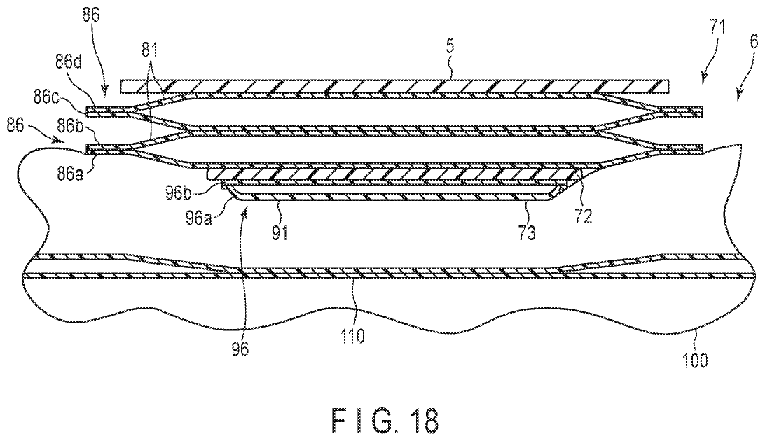

FIG. 15 is a cross-sectional diagram schematically showing configurations of a curler 5 and the cuff structure 6 of the blood pressure measuring device 1 taken along line XV-XV in FIG. 14. FIG. 16 is a cross-sectional view of configurations of the curler 5 and the cuff structure 6, taken along line XVI-XVI in FIG. 14. FIGS. 17 and 18 are a side view and sectional view, respectively, which schematically show an example in which a pressing cuff 71 and the sensing cuff 73 of the cuff structure 6 are inflated. In FIG. 15, the curler 5 and the cuff structure 6 are schematically shown in a linear shape for convenience of explanation; however, these components are in a bent shape when provided in the blood pressure measuring device 1.

The blood pressure measuring device 1 is an electronic blood pressure measuring device worn on a living body. In the present embodiment, an electronic blood pressure measuring device in the form of a wearable device worn on a wrist 100 of a living body will be described. As shown in FIGS. 1 to 18, the blood pressure measuring device 1 includes: the device main body 3; the belt 4; the curler 5; the cuff structure 6 with the pressing cuff 71 and the sensing cuff 73; and a fluid circuit 7. In the present embodiment, the pressing cuff 71 is an example of the “cuff” of the present invention.

As shown in FIGS. 1 to 18, the device main body 3 includes a case 11, a display 12, an operation unit 13, a pump 14, a flow path section 15, an on-off valve 16, a pressure sensor 17, a power supply unit 18, a vibration motor 19, and a control substrate 20. The device main body 3 is a supply device that supplies a fluid to the pressing cuff 71 by using the pump 14, the on-off valve 16, the pressure sensor 17, the control substrate 20, and the like.

The case 11 includes an outer case 31; a windshield 32 that covers an upper opening of the outer case 31; a base 33 provided in a lower part of the inside of the outer case 31; a flow path cover 34 that covers a part of a back surface of the base 33; and a back cover 35 that covers a lower side of the outer case 31. The case 11 also includes a flow path tube 36 constituting a part of the fluid circuit 7.

The outer case 31 is formed in a cylindrical shape. The outer case 31 includes: pairs of lugs 31a provided at symmetrical positions in the circumferential direction of the outer peripheral surface; and spring bars 31b respectively provided between the paired lugs 31a. The windshield 32 is a circular glass plate.

The base 33 holds the display 12, the operation unit 13, the pump 14, the on-off valve 16, the pressure sensor 17, the power supply unit 18, the vibration motor 19, and the control substrate 20. The base 33 forms a part of the flow path section 15.

The flow path cover 34 is fixed to a back surface of the base 33, which is an outer surface of the base 33 on the back cover 35 side. A groove is provided in one or both of the base 33 and the flow path cover 34, thereby forming a part of the flow path section 15.

The back cover 35 covers an end of the outer case 31 on the living body side. The back cover 35 is fixed to an end of the outer case 31 or the base 33 on the living body side by, for example, four screws 35a or the like.

The flow path tube 36 forms a part of the flow path section 15. The flow path tube 36 connects, for example, the on-off valve 16 and a part of the base 33 constituting the flow path section 15.

The display 12 is disposed on the base 33 of the outer case 31 and directly below the windshield 32. The display 12 is electrically connected to the control substrate 20. The display 12 is, for example, a liquid crystal display or an organic electroluminescence display. The display 12 displays various kinds of information including date and time, and measurement results of blood pressure values, such as systolic blood pressure and diastolic blood pressure, a heart rate, and the like.

The operation unit 13 is configured to allow a user to input a command. For example, the operation unit 13 includes: a plurality of buttons 41 provided to the case 11; a sensor 42 that detects an operation of the buttons 41; and a touch panel 43 provided to the display 12 or the windshield 32. The operation unit 13 is operated by a user to convert a command into an electric signal. The sensor 42 and the touch panel 43 are electrically connected to the control substrate 20 and output an electric signal to the control substrate 20.

For example, three buttons 41 are provided. The buttons 41 are supported by the base 33 and protrude from the outer peripheral surface of the outer case 31. The plurality of buttons 41 and the plurality of sensors 42 are supported by the base 33. For example, the touch panel 43 is provided integrally to the windshield 32.

The pump 14 is, for example, a piezoelectric pump. The pump 14 compresses the air and supplies the compressed air to the cuff structure 6 via the flow path section 15. The pump 14 is electrically connected to a controller 55.

The flow path section 15 is an air flow path formed of a groove or the like provided in the flow path cover 34 that covers the back cover 35 side of the base 33 and the main surface of the base 33 on the back cover 35 side. The flow path section 15 forms a flow path leading from the pump 14 to the pressing cuff 71, and a flow path leading from the pump 14 to the sensing cuff 73. The flow path section 15 also forms a flow path leading from the pressing cuff 71 to the atmosphere, and a flow path leading from the sensing cuff 73 to the atmosphere. The flow path cover 34 includes a connected portion 34a to which the pressing cuff 71 and the sensing cuff 73 are connected. The connected portion 34a is, for example, a cylindrical nozzle provided to the flow path cover 34.

The on-off valve 16 opens and closes a part of the flow path section 15. For example, a plurality of on-off valves 16 are provided, and selectively open and close the flow path leading from the pump 14 to the pressing cuff 71; the flow path leading from the pump 14 to the sensing cuff 73; the flow path leading from the pressing cuff 71 to the atmosphere; and the flow path leading from the sensing cuff 73 to the atmosphere, depending on the combination of the opening and closing of the on-off valves 16. For example, two on-off valves 16 are used.

The pressure sensor 17 detects the pressure of the pressing cuff 71 and the sensing cuff 73. The pressure sensor 17 is electrically connected to the control substrate 20. The pressure sensor 17 is electrically connected to the control substrate 20, converts the detected pressure into an electric signal, and outputs the electric signal to the control substrate 20. For example, the pressure sensor 17 is provided in the flow path leading from the pump 14 to the pressing cuff 71 and the flow path leading from the pump 14 to the sensing cuff 73. Since these flow paths are continuous with the pressing cuff 71 and the sensing cuff 73, the pressures in these flow paths become the pressures in the internal spaces of the pressing cuff 71 and the sensing cuff 73.

The power supply unit 18 is, for example, a secondary battery such as a lithium ion battery. The power supply unit 18 is electrically connected to the control substrate 20. The power supply unit 18 supplies power to the control substrate 20.

As shown in FIGS. 4 and 12, the control substrate 20 includes, for example, a substrate 51, an acceleration sensor 52, a communication unit 53, a storage 54, and the controller 55. The control substrate 20 is configured by mounting the acceleration sensor 52, the communication unit 53, the storage 54, and the controller 55 on the substrate 51.

The substrate 51 is fixed to the base 33 of the case 11 by a screw or the like.

The acceleration sensor 52 is, for example, a three-axis acceleration sensor. The acceleration sensor 52 outputs, to the controller 55, acceleration signals representing accelerations of the device main body 3 in three directions that are orthogonal to one another. For example, the acceleration sensor 52 is used to measure the amount of activity of the living body wearing the blood pressure measuring device 1 based on the detected accelerations.

The communication unit 53 is configured to be able to transmit and receive information to and from an external device in a wireless or wired manner. For example, the communication unit 53 transmits information controlled by the controller 55 and information such as measured blood pressure values, pulse, and the like to an external device via a network, and receives a program for software update, etc., from the external device via the network to transmit the program, etc., to the controller.

In the present embodiment, the network is, for example, the Internet, but is not limited thereto. The network may be a network such as a local area network (LAN) provided in a hospital, or direct communication with an external device using, for example, a cable having a terminal of a predetermined standard such as a USB may be adopted. Therefore, the communication unit 53 may include a plurality of wireless antennas, micro USB connectors, and the like.

The storage 54 stores in advance program data for controlling the entire blood pressure measuring device 1 and the fluid circuit 7, setting data for the setting of various functions of the blood pressure measuring device 1, calculation data for the calculation of blood pressure values and a pulse from a pressure measured by the pressure sensor 17, and the like. The storage 54 also stores information such as measured blood pressure values and pulse.

The controller 55 is formed of one or more CPUs, and controls the operation of the entire blood pressure measuring device 1 and the operation of the fluid circuit 7. The controller 55 is electrically connected to the display 12, the operation unit 13, the pump 14, the on-off valves 16, and the pressure sensors 17, and supplies electric power. Also, the controller 55 controls the operations of the display 12, the pump 14, and the on-off valves 16 based on the electric signals output from the operation unit 13 and the pressure sensor 17.

For example, the controller 55 includes a main CPU 56 that controls the operation of the entire blood pressure measuring device 1 and a subordinate CPU 57 that controls the operation of the fluid circuit 7, as shown in FIG. 4. For example, when a command to measure blood pressure is input from the operation unit 13, the subordinate CPU 57 drives the pump 14 and the on-off valves 16 to send compressed air to the pressing cuff 71 and the sensing cuff 73.

The subordinate CPU 57 also controls the driving and stoppage of the pump 14 and the opening and closing of the on-off valves 16 based on the electric signal output from the pressure sensor 17, selectively sends compressed air to the pressing cuff 71 and the sensing cuff 73, and selectively pressurizes the pressing cuff 71 and the sensing cuff 73. The main CPU 56 obtains measurement results of blood pressure values, such as systolic blood pressure and diastolic blood pressure, a heart rate, and the like from the electric signal output from the pressure sensor 17, and outputs an image signal corresponding to the measurement results to the display 12.

As shown in FIGS. 1 to 13, the belt 4 includes a first belt portion 61 provided to one of the pairs of lugs 31a and the spring bar 31b, and a second belt portion 62 provided to the other pair of lugs 31a and the spring bar 31b.

The first belt portion 61 is a so-called “parent” and is made of a resin material. The first belt is provided on one side of the curler 5, for example, to cover a part of the curler 5. The first belt portion 61 is formed in a band shape bent along the outer periphery of the curler 5.

The first belt portion 61 includes a first hole 61a provided at one end and perpendicular to the longitudinal direction of the first belt portion 61, a second hole 61b provided at the other end and perpendicular to the longitudinal direction of the first belt portion 61, and a buckle 61c provided in the second hole 61b. The first hole 61a has an inner diameter so that the spring bar 31b can be inserted thereinto and the first belt portion 61 can rotate with respect to the spring bar 31b. That is, the first hole 61a is disposed between the paired lugs 31a and at the spring bar 31b, so that the first belt portion 61 is rotatably held by the outer case 31. The second hole 61b is provided at a distal end of the first belt portion 61.

As shown in FIG. 6, the first belt portion 61 includes a cover layer 63, a first insert layer 64, and a second insert layer 65. The first belt portion 61 is formed in a bent shape along the outer periphery of the living body.

The cover layer 63 is made of, for example, a thermosetting resin. The cover layer 63 is made of, for example, an elastically deformable resin material having flexibility. For example, a thermosetting elastomer is a type of thermosetting resin, and silicone resin and fluororesin are types of thermosetting elastomer.

The first insert layer 64 is disposed within the cover layer 63. The first insert layer 64 is formed of a first insert material 64A. The first insert material 64A is made of a high-tensile material. The first insert material 64A is, for example, a high-tensile sheet formed of a high-tensile material having a tensile strength higher than that of the resin material forming the cover layer 63. Specifically, the tensile strength of the high-tensile material in the circumferential direction of the living body is higher than that of the thermosetting resin forming the cover layer 63. Examples of the high-tensile material include high-strength polyarylate (Vectran) fibers, liquid crystal polymers, PET resins, and PEN resins. The first insert material 64A is formed in a mesh shape or a film shape. The first insert layer 64 is slightly shorter than the cover layer 63 in both the width direction and the circumferential direction, and covered with the cover layer 63. The first insert material 64A is disposed, for example, on the outer side of the curve of the second insert material 65A.

The second insert layer 65 is formed of a second insert material 65A disposed within the cover layer 63. The second insert material 65A is, for example, a resin sheet made of a thermoplastic resin. The second insert layer 65 is stacked on the inner side of the curved shape of the first insert layer 64. The second insert layer 65 is slightly shorter than the cover layer 63 in both the width direction and the circumferential direction, and covered with the cover layer 63. The second insert layer 65 has a thickness of about 1.0 mm.

The buckle 61c includes a rectangular frame-shaped body 61d and a prodding stick 61e rotatably attached to the frame-shaped body 61d. One side of the frame-shaped body 61d to which the prodding stick 61e is attached is inserted into the second hole 61b, so that the frame-shaped body 61d is rotatably attached with respect to the first belt portion 61.

Next, a method of manufacturing the belt 4, which is part of a method of manufacturing the blood pressure measuring device 1 according to an embodiment, will be described with reference to FIGS. 5 to 9. The method of manufacturing the blood pressure measuring device 1 includes a preform forming step and a bending step as a method of manufacturing the first belt portion 61.

In the preform forming step, firstly, a base part 63a is formed, as shown in ST11 of FIG. 7. The resin forming the cover layer 63 is molded by application of heat using a first mold 101 for the base part 63a, to form a band-like base part 63a having a predetermined shape. Next, the first insert material 64A and the second insert material 65A are placed on the base part 63a, as shown in ST12 of FIG. 7. Then, a linearly-extending band-shaped preform 61A is formed, as shown in ST13. Specifically, by using a mold 102 corresponding to the band-shaped preform 61A, the thermosetting resin forming the cover layer 63 is disposed on the base part 63a and the insert materials 64 and 65 to perform insert molding. At this time, heating is performed to a predetermined temperature to cure the thermosetting resin forming the cover layer 63 and soften the second insert material 65A, thereby forming the preform 61A.

As shown in ST13 of FIG. 7 as well as in FIG. 8, the preform 61A has a linearly-extending band shape immediately after the insert molding.

Next, for the bending step, the preform 61A is housed in a mold 103 having a predetermined shape along the outer periphery of the living body and is bent, as shown in ST14. For example, the bending step involves a curing step in which the preform 61A is bent and cooled to a temperature lower than the temperature at which the insert molding is performed, so that the insert material is cured. As a result, the preform 61A is bent into a predetermined shape. The bent first belt portion 61 is removed from the mold 103, and the buckle 61c is attached to complete the first belt portion 61.

The cover layer 63 and the second insert material 65 may be formed of a thermoplastic resin as long as the curing timing differs depending on the heating temperature. For example, by using resins having different softening points and curing points, the properties, such as bendability, of both the cover layer 63 and the second insert material 65 can be made different even at the same temperature.

In addition to the above, it is also possible to form and bend the preform 61A in the mold 102 by, for example, using the mold 102 having a bent shape in the ST13.

The second belt portion 62 is a so-called “pointed end”, and formed in a band shape having a width that allows the second belt portion 62 to be inserted into the frame-shaped body 61d. The second belt portion 62 includes a plurality of small holes 62a into which the prodding stick 61e is inserted. The second belt portion 62 also includes a third hole 62b provided at one end of the second belt portion 62 and perpendicular to the longitudinal direction of the second belt portion 62. The third hole 62b has an inner diameter so that the spring bar 31b can be inserted thereinto and that the second belt portion 62 can rotate with respect to the spring bar 31b. That is, the third hole 62b is disposed between the paired lugs 31a and at the spring bar 31b, so that the second belt portion 62 is rotatably held by the outer case 31.

As shown in FIG. 9, the second belt portion 62 includes a cover layer 63 and a first insert layer 64. As an example, the cover layer 63 and the first insert layer 64 are made of the same material as that of the first belt portion 61. Specifically, the second belt portion 62 does not include the second insert layer 65 included in the first belt portion 61.

The cover layer 63 is made of, for example, a thermosetting resin. The cover layer 63 is made of, for example, an elastically deformable resin material having flexibility.

The first insert layer 64 is disposed within the cover layer 63. The first insert layer 64 is formed of the first insert material 64A. The first insert material 64A is made of a high-tensile material. The first insert material 64A is, for example, a high-tensile sheet formed of a high-tensile material having a tensile strength higher than that of the resin material forming the cover layer. Specifically, the tensile strength of the high-tensile material in the circumferential direction of the living body is higher than that of the thermosetting resin forming the cover layer 63. Examples of the high-tensile material include Vectran fibers, liquid crystal polymers, PET resins, and PEN resins. The first insert material 64A is formed in a mesh shape or a film shape. The first insert layer 64 is slightly shorter than the cover layer 63 in both the width direction and the circumferential direction, and covered with the cover layer 63. The first insert layer 64 is disposed, for example, at a position closer to the outer periphery of the curve than the central position in the thickness direction of the cover layer 63.

In the method of forming the second belt portion 62, firstly, the resin forming the cover layer is thermally molded using the first mold 101, so as to form a band-like base part 63a having a predetermined shape by insert molding, as shown in ST11 of FIG. 7, in the same manner as in the preform forming step for the first belt portion 61, for example. Next, the first insert material 64A is placed on the base part 63a, as shown in ST12 of FIG. 7, and a thermosetting resin is disposed on both the base part 63a and the insert material 64 to perform insert molding, as shown in ST13 of FIG. 7, thereby forming the remaining portion of the cover layer 63. As a result, the second belt portion 62 is formed.

The belt 4 described above forms an annular shape along the circumferential direction of the wrist 100 together with the outer case 31 as the second belt portion 62 is inserted into the frame-shaped body 61d and the prodding stick 61e is inserted into the small hole 62a, thereby integrally connecting the first belt portion 61 and the second belt portion 62 to each other.

The curler 5 is made of a resin material and has a band shape bent along the circumferential direction of the wrist. For example, one end of the curler 5 is fixed between the back cover 35 and the base 33 as well as the flow path cover 34 of the device main body 3, and the other end of the curler 5 is close to the device main body 3. As shown in FIG. 10, the curler 5 may be configured so that the curler 5 is fixed to the outer surface of the back cover 35, that one end of the curler 5 protrudes from a side of the back cover 35 closer to one of the pairs of lugs 31a, and that the other end of the curler 5 protrudes from a side of the back cover 35 closer to the other pair of lugs 31a and extends to a position adjacent to one end of the curler 5.

As shown in FIGS. 1 to 3, the curler 5 is made of a resin material having a shape bent along the circumferential direction of the wrist 100, for example, in a side view from a direction perpendicular to the circumferential direction of the wrist, in other words, the longitudinal direction of the wrist. For example, the curler 5 extends from the device main body to the palmar side through the dorsal side of the wrist and one side of the wrist, and extends toward the center of the other side of the wrist. That is, the curler 5 bends along the circumferential direction of the wrist and thereby extends over most parts of the wrist 100 in the circumferential direction of the wrist 100, and both ends of the curler 5 are separated from each other by a predetermined interval.

The curler 5 has a hardness encompassing both flexibility and shape-retaining capability. The “flexibility” means that the curler 5 deforms in the radial direction when an external force is applied to the curler 5, and means that when the curler 5 is pressed by the belt 4, for example, the curler 5 deforms so as to approach the wrist, conform to the shape of the wrist, or trace the shape of the wrist, as viewed from a side of the curler 5. The “shape-retaining capability” means that the curler 5 can maintain a pre-formed shape when no external force is applied thereto; and in the present embodiment, it means that the curler 5 can maintain a shape bent along the circumferential direction of the wrist. The curler 5 is made of a resin material. For example, the curler 5 is made of polypropylene and has a thickness of about 1 mm. The curler 5 holds the cuff structure 6 along the inner surface shape of the curler 5.

As shown in FIGS. 1 to 3 and 14 to 16, the cuff structure 6 includes the pressing cuff 71, the back plate 72, and the sensing cuff 73. The cuff structure 6 is configured so that the pressing cuff 71, the back plate 72, and the sensing cuff 73 are stacked and integrally formed. The cuff structure 6 is fixed to the inner surface of the curler 5.

The pressing cuff 71 is an example of the cuff. The pressing cuff 71 is fluidly connected to the pump 14 via the flow path section 15. The pressing cuff 71 is inflated to press the back plate 72 and the sensing cuff 73 toward the living body. The pressing cuff 71 includes a plurality of air bags 81, a tube 82 communicating with the air bags 81, and a connection portion 83 provided at a distal end of the tube 82.

The air bag 81 is a bag-shaped structure. Since the blood pressure measuring device 1 is configured to use the air with the pump 14 in the present embodiment, an air bag will be described. However, when a fluid other than the air is used, the bag-shaped structure may be a fluid bag such as a liquid bag.

The plurality of air bags 81 are stacked and fluidly communicate with each other in the stacking direction. As a specific example, the pressing cuff 71 includes: two layers of air bags 81 fluidly communicating with each other in the stacking direction; a tube 82 provided on one end of one of the air bags 81 in the longitudinal direction; and a connection portion 83 provided at a distal end of the tube 82.

The pressing cuff 71 is configured so that the main surface of one of the air bags 81 is fixed to the inner surface of the curler 5. For example, the pressing cuff 71 is attached to the inner surface of the curler 5 by a double-sided tape, an adhesive, or the like.

The two layers of air bags 81 are formed in a rectangular shape elongated in one direction. The air bag 81 is formed by, for example, combining two sheet members 86 elongated in one direction and welding the edges thereof by heat. As a specific example, the two layers of air bags 81 include, from the living body side: a first sheet member 86a; a second sheet member 86b forming the first layer of air bag 81 with the first sheet member 86a; a third sheet member 86c integrally bonded to the second sheet member 86b; and a fourth sheet member 86d forming the second layer of air bag 81 with the third sheet member 86c, as shown in FIGS. 14 to 16.

The first sheet member 86a and the second sheet member 86b form the air bag 81 by the welding of the peripheral edges of the four sides of the sheet members. The second sheet member 86b and the third sheet member 86c are disposed to face each other, and each include a plurality of openings 86b1 and 86c1 that fluidly connect the two air bags 81. The fourth sheet member 86d has an adhesive layer or a double-sided tape on the outer surface thereof on the curler 5 side, and is attached to the curler 5 by the adhesive layer or the double-sided tape.

The third sheet member 86c and the fourth sheet member 86d form the air bag 81 by the welding of the peripheral edges of the four sides of the sheet members. Also, for example, the tube 82 fluidly continuous with the internal space of the air bag 81 is disposed on one side of the third sheet member 86c and the fourth sheet member 86d, and is fixed by welding. For example, the third sheet member 86c and the fourth sheet member 86d form the air bag 81 by the welding of the peripheral edges of the four sides of the sheet members with the tube 82 disposed between the third sheet member 86c and the fourth sheet member 86d, thereby integrally welding the tube 82 thereto.

The tube 82 is connected to one of the two layers of air bags 81, and is provided at one end in the longitudinal direction of the air bag 81. As a specific example, the tube 82 is provided at an end on the curler 5 side of the two layers of air bags 81 and close to the device main body 3. The tube 82 includes the connection portion 83 at its distal end. The tube 82 forms a flow path between the device main body 3 and the air bag 81 in the fluid circuit 7. The connection portion 83 is connected to the connected portion 34a of the flow path cover 34. The connection portion 83 is, for example, a nipple.

The back plate 72 is attached to the outer surface 86a1 of the first sheet member 86a of the pressing cuff 71 by an adhesive layer, a double-sided tape, or the like. The back plate 72 is made of a resin material and formed in a plate shape. For example, the back plate 72 is made of polypropylene and formed in a plate shape having a thickness of about 1 mm. The back plate 72 has shape traceability.

The “shape traceability” refers to a function that allows the back plate 72 to deform so as to trace the shape of a contacted portion of the wrist 100 to be placed; the “contacted portion of the wrist 100” refers to a region that comes into contact with the back plate 72; and the “contact” includes both direct and indirect contact.

Therefore, the shape traceability is a function of deforming the back plate 72 provided to the pressing cuff 71 or the back plate 72 provided between the pressing cuff 71 and the sensing cuff 73 to such an extent that the back plate 72 itself or the sensing cuff 73 provided to the back plate 72 conforms to the wrist 100 or comes into close contact with the wrist 100 along the wrist 100.

For example, the back plate 72 includes a plurality of grooves 72a on both main surfaces of the back plate 72 at positions facing each other and at equal distances in the longitudinal direction of the back plate 72. As a result, the portion of the back plate 72 having the grooves 72a is thinner than the portion of the back plate 72 without the grooves 72a, and is thus easily deformed. Accordingly, the back plate 72 has shape traceability of deforming in accordance with the shape of the wrist 100. The back plate 72 is formed to have a length covering the palmar side of the wrist 100. The back plate 72 transmits the pressing force from the pressing cuff 71 to the main surface of the sensing cuff 73 on the back plate 72 side, in a state of conforming to the shape of the wrist 100.

The sensing cuff 73 is fixed to the main surface of the back plate 72 on the living body side. As shown in FIG. 17, the sensing cuff 73 directly contacts the region of the wrist 100 where arteries exist. The sensing cuff 73 is formed in the same shape as that of the back plate 72 or in a shape smaller than that of the back plate 72, in the longitudinal direction and the width direction of the back plate 72. The sensing cuff 73 is inflated to compress a region of the wrist 100 on the palmar side where the arteries 110 exist. The sensing cuff 73 is pressed toward the living body by the inflated pressing cuff 71 via the back plate 72.

As a specific example, the sensing cuff 73 includes one air bag 91, a tube 92 communicating with the air bag 91, and a connection portion 93 provided at a distal end of the tube 92. The sensing cuff 73 is configured so that one of the main surfaces of the air bag 91 is fixed to the back plate 72. For example, the sensing cuff 73 is attached to the main surface of the back plate 72 on the living body side by a double-sided tape, an adhesive layer, or the like.

The air bag 91 is a bag-shaped structure. Since the blood pressure measuring device 1 is configured to use the air with the pump 14 in the present embodiment, an air bag will be described. However, when a fluid other than the air is used, the bag-shaped structure may be a liquid bag or the like. A plurality of air bags 91 described above are stacked and fluidly communicate with each other in the stacking direction.

The air bag 91 is formed in a rectangular shape elongated in one direction. The air bag 91 is formed by, for example, combining two sheet members elongated in one direction and the welding of the edges thereof by heat. As a specific example, the air bag 91 includes a fifth sheet member 96a and a sixth sheet member 96b from the living body side, as shown in FIGS. 9 and 10.

For example, the fifth sheet member 96a and the sixth sheet member 96b are configured so that the tube 92 fluidly continuous with the internal space of the air bag 91 is disposed on one side of the fifth sheet member 96a and the sixth sheet member 96b, and is fixed by welding. For example, the fifth sheet member 96a and the sixth sheet member 96b form the air bag 91 by the welding of the peripheral edges of the four sides of the sheet members with the tube 92 disposed between the fifth sheet member 96a and the sixth sheet member 96b, thereby integrally welding the tube 92 thereto.

The tube 92 is provided at one end in the longitudinal direction of the air bag 91. As a specific example, the tube 92 is provided at an end of the air bag 91 close to the device main body 3. The tube 92 includes the connection portion 93 at its distal end. The tube 92 forms a flow path between the device main body 3 and the air bag 91 in the fluid circuit 7. The connection portion 93 is connected to the connected portion 34a of the flow path cover 34. The connection portion 93 is, for example, a nipple.

The sheet members 86 and 96 forming the pressing cuff 71 and the sensing cuff 73 are made of a thermoplastic elastomer. For example, thermoplastic polyurethane resin (hereinafter referred to as “TPU”), vinyl chloride resin, ethylene-vinyl acetate resin, thermoplastic polystyrene resin, thermoplastic polyolefin resin, thermoplastic polyester resin, and thermoplastic polyamide resin may be used as the thermoplastic elastomer forming the sheet members 86 and 96. TPU is preferably used as the thermoplastic elastomer. The sheet member may have a single-layer structure or a multi-layer structure.

The sheet members 86 and 96 are not limited to the thermoplastic elastomer, and may be a thermosetting elastomer such as silicone or a combination of a thermoplastic elastomer (for example, TPU) and a thermosetting elastomer (for example, silicone).

When a thermoplastic elastomer is used for the sheet members 86 and 96, a molding method such as T-die extrusion molding or injection molding is adopted, and when a thermosetting elastomer is used for the sheet members 86 and 96, a molding method such as mold casting molding is adopted. The sheet members are molded by the molding method and thereafter sized into a predetermined shape. Then, the sized pieces are bonded by adhesion, welding, or the like to form the air bags 81 and 91 being bag-shaped structures. As a bonding method, a high-frequency welder or laser welding is used when a thermoplastic elastomer is used, and a molecular adhesive is used when a thermosetting elastomer is used.

The fluid circuit 7 is formed of the case 11, the pump 14, the flow path section 15, the on-off valve 16, the pressure sensor 17, the pressing cuff 71, and the sensing cuff 73. Hereinafter, a specific example of the fluid circuit 7 will be described in which the two on-off valves 16 used in the fluid circuit 7 are referred to as a “first on-off valve 16A” and a “second on-off valve 16B”, and the two pressure sensors 17 used in the fluid circuit 7 are referred to as a “first pressure sensor 17A” and a “second pressure sensor 17B”.

As shown in FIG. 4, the fluid circuit 7 includes, for example, a first flow path 7a which continues from the pump 14 to the pressing cuff 71, a second flow path 7b which is formed by branching a middle portion of the first flow path 7a and continues from the pump 14 to the sensing cuff 73, and a third flow path 7c which connects the first flow path 7a and the atmosphere. The first flow path 7a includes the first pressure sensor 17A. The first on-off valve 16A is provided between the first flow path 7a and the second flow path 7b. The second flow path 7b includes the second pressure sensor 17B. The second on-off valve 16B is provided between the first flow path 7a and the third flow path 7c.

In the fluid circuit 7 described above, when the first on-off valve 16A and the second on-off valve 16B are closed, only the first flow path 7a is connected to the pump 14, and the pump 14 and the pressing cuff 71 are fluidly connected. In the fluid circuit 7, when the first on-off valve 16A is opened and the second on-off valve 16B is closed, the first flow path 7a and the second flow path 7b are connected, and the pump 14 and the pressing cuff 71, and the pump 14 and the sensing cuff 73 are fluidly connected. In the fluid circuit 7, when the first on-off valve 16A is closed and the second on-off valve 16B is closed, the first flow path 7a and the third flow path 7c are connected, and the pressing cuff 71 and the atmosphere are fluidly connected. In the fluid circuit 7, when the first on-off valve 16A and the second on-off valve 16B are opened, the first flow path 7a, the second flow path 7b, and the third flow path 7c are connected, and the pressing cuff 71, the sensing cuff 73, and the atmosphere are fluidly connected.

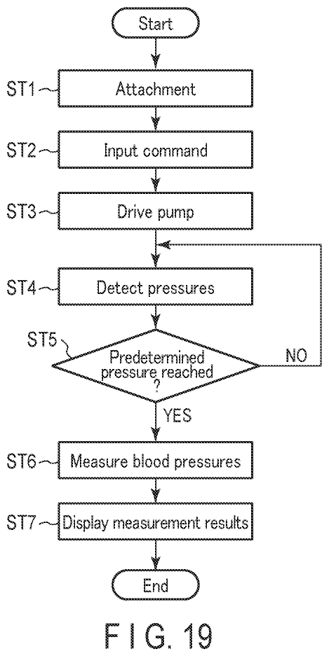

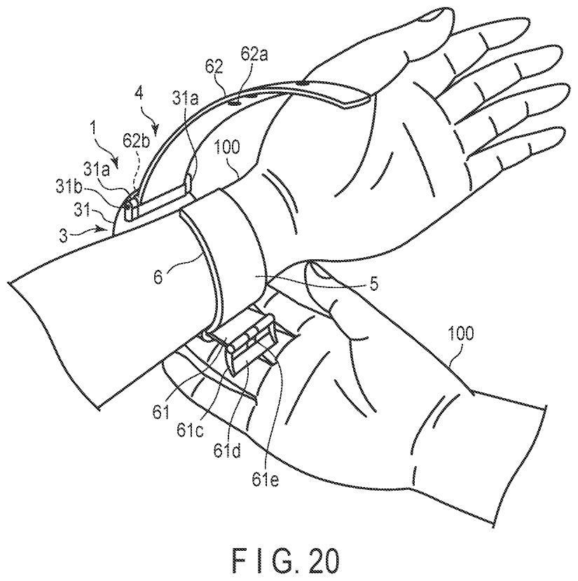

Next, an example of measurement of blood pressure values using the blood pressure measuring device 1 will be described with reference to FIGS. 19 to 22. FIG. 19 is a flowchart showing an example of blood pressure measurement using the blood pressure measuring device 1, and shows both the operation of a user and the operation of the controller 55. FIGS. 20 to 22 show an example in which the user wears the blood pressure measuring device 1 on the wrist 100.

First, the user wears the blood pressure measuring device 1 on the wrist 100 (step ST1). As a specific example, the user, for example, inserts one of the wrists 100 into the curler 5, as shown in FIG. 20.

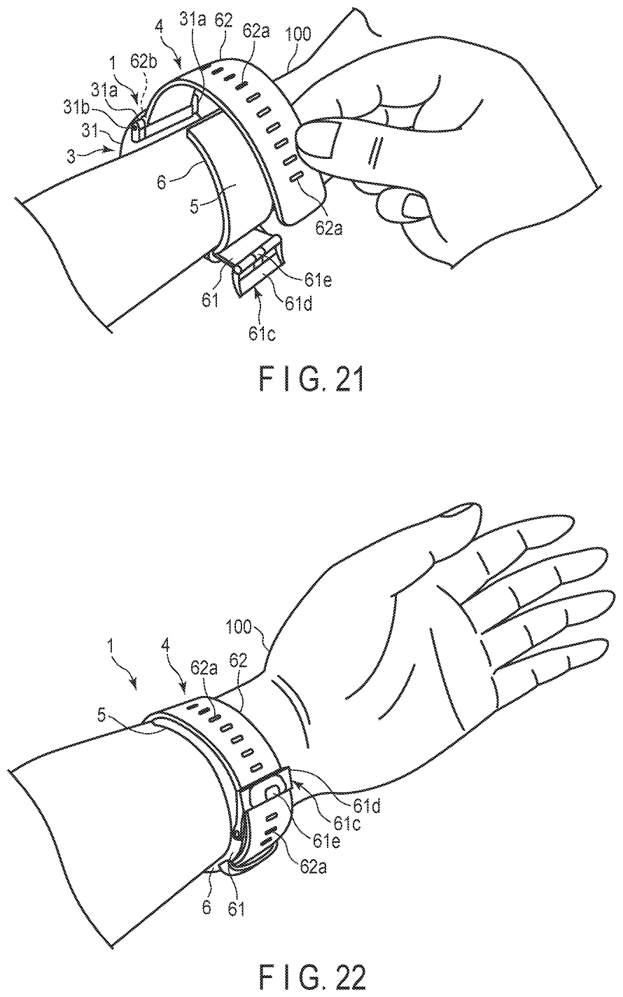

At this time, since the device main body 3 and the sensing cuff 73 are disposed at positions of the curler 5 opposed to each other in the blood pressure measuring device 1, the sensing cuff 73 is disposed in a region of the wrist 100 on the palmar side where the arteries 110 exist. Thus, the device main body 3 is disposed on the dorsal side of the wrist 100. Next, the user passes the second belt portion 62 through the frame-shaped body 61d of the buckle 61c of the first belt portion 61 using the hand opposite to the hand on which the blood pressure measuring device 1 is placed, as shown in FIG. 21. Then, the user pulls the second belt portion 62, brings the member on the inner peripheral surface side of the curler 5, that is, the cuff structure 6, into close contact with the wrist 100, and inserts the prodding stick 61e into the small hole 62a. As a result, the first belt portion 61 and the second belt portion 62 are connected, and the blood pressure measuring device 1 is worn on the wrist 100, as shown in FIG. 22.

Next, the user operates the operation unit 13 to input a command corresponding to initiation of measurement of blood pressure values. The operation unit 13 in which the input operation of the command has been performed outputs an electric signal corresponding to initiation of measurement to the controller 55 (step ST2). Upon receiving the electric signal, the controller 55, for example, opens the first on-off valve 16A and closes the second on-off valve 16B, drives the pump 14, and supplies compressed air to the pressing cuff 71 and the sensing cuff 73 through the first flow path 7a and the second flow path 7b (step ST3). Thereby, the pressing cuff 71 and the sensing cuff 73 start to inflate.

The first pressure sensor 17A and the second pressure sensor 17B detect the pressures of the pressing cuff 71 and the sensing cuff 73, and output electric signals corresponding to the detected pressures to the controller 55 (step ST4). Based on the received electric signals, the controller 55 determines whether or not the pressures in the internal spaces of the pressing cuff 71 and the sensing cuff 73 reach a predetermined pressure for measuring blood pressure (step ST5). For example, when the internal pressure of the pressing cuff 71 does not reach the predetermined pressure and the internal pressure of the sensing cuff 73 reaches the predetermined pressure, the controller 55 closes the first on-off valve 16A and supplies compressed air through the first flow path 7a.

When both the internal pressure of the pressing cuff 71 and the internal pressure of the sensing cuff 73 reach the predetermined pressure, the controller 55 stops driving the pump 14 (YES in step ST5). At this time, the pressing cuff 71 is sufficiently inflated, and the inflated pressing cuff 71 presses the wrist 100 and the back plate 72, as shown in FIG. 17.

Further, the sensing cuff 73 is sufficiently inflated and pressed toward the wrist 100 by the back plate 72 pressed by the pressing cuff 71. Therefore, the sensing cuff 73 presses the arteries 110 in the wrist 100 to occlude the arteries 110, as shown in FIG. 18.

Thereafter, the controller 55 controls the second on-off valve 16B to repeatedly open and close the second on-off valve 16B or adjust the opening degree of the second on-off valve 16B, thereby increasing the pressure in the internal space of the pressing cuff 71. Based on the electric signal output from the second pressure sensor 17B in the pressurization process, the controller 55 obtains measurement results of blood pressure values, such as systolic blood pressure and diastolic blood pressure, a heart rate, and the like.

The example in which the timing of opening and closing the first on-off valve 16A and the second on-off valve 16B at the time of blood pressure measurement may be suitably set, and the controller 55 calculates blood pressure in the process of pressurizing the pressing cuff 71, is described above. However, blood pressure may be calculated in the process of depressurizing the pressing cuff 71 or in both the processes of pressurizing and depressurizing the pressing cuff 71. The controller 55 then outputs an image signal corresponding to the obtained measurement results to the display 12.

Upon receiving the image signal, the display 12 displays the measurement results on a screen. The user checks the measurement results by viewing the display 12. After the measurement is completed, the user removes the prodding stick 61e from the small hole 62a, removes the second belt portion 62 from the frame-shaped body 61d, and removes the wrist 100 from the curler 5, thereby removing the blood pressure measuring device 1 from the wrist 100.

In the blood pressure measuring device 1 according to the embodiment configured as described above, the belt 4 provided on the outer periphery of the cuff structure 6 inflated by a fluid includes the high-tensile material having a high tensile strength; therefore, it is possible to suppress elongation of the belt 4 even when a stress is applied in a direction in which the belt is pulled due to the inflation of the cuff. Also, since the cover layer 63 forming the outer surface of the belt 4 is formed of a resin material, the flexibility of the resin material can ensure ease of attachment in the attachment process. That is, if the entire belt 4 is made of a material having a high degree of hardness in order to reduce the likelihood of stretching, the flexibility at the time of attachment is impaired; however, with a multilayer structure in which a high-tensile material having a tensile strength higher than that of the cover layer 63 is disposed in the resin cover layer 63 forming the outer surface, both ease of attachment and reduced likelihood of stretching can be achieved. Therefore, it is possible to implement blood pressure measurement with high accuracy by suppressing the elongation of the belt 4 while maintaining close contact when the cuff is inflated at the time of blood pressure measurement.

Since the first belt portion 61 is formed in a bent shape, it demonstrates high workability when the belt portion is wound around and attached to the living body. That is, since a fastener such as the buckle 61c is positioned at a desired position near the living body, work of positioning is rendered unnecessary.

Therefore, it is possible to provide the belt 4 that is prevented from being stretched and has a good level of wearability.

Since the high-tensile material is formed in a band shape and a film shape by a mesh, a film or the like, the belt can be made in a thin and lightweight form. Also, if the high-tensile material is formed in a mesh shape, the cover layer 63 and the first insert layer 64 will have good bonding properties and are less likely to peel off. Further, by disposing the first insert material 64A on the outer side of the curve of the second insert material 65A, wrinkles are less likely to be generated when the preform processing is performed.

According to the method of manufacturing the blood pressure measuring device 1, insert molding is performed to form a simple linear band shape, die cutting is executed, and the resultant is then accommodated in a mold having a desired shape and bent. Therefore, the molding process and the configuration of the mold can be simplified.

That is, since the cover layer 63 of the first belt portion 61 forming the outer layer is made of a thermosetting resin and the insert layer 64 of the first belt portion 61 on the inside is made of a thermoplastic resin, the curing of the cover layer 63 and the softening of the insert layer 64 can be implemented in a single process by adjusting the temperature at which insert molding is performed and the temperature at which cooling is performed. Furthermore, since the bending into a desired shape is performed by cooling, the bending process can be easily performed.

As described above, the belt 4 provided in the blood pressure measuring device 1 can suppress elongation of the belt 4 even when a stress pulling the belt 4 is applied in the circumferential direction of the living body due to the inflation of the cuff. Therefore, high measurement accuracy can be maintained.

In addition, since the linear preform is formed once and the thermosetting resin after heating is die-cut, the die-cutting process can be easily performed and there are few restrictions in the shape of the mold and the die-cutting process. Accordingly, surface processing, such as emboss processing or concavo-convex processing, can be easily performed on the surface of the first belt portion 61, for example, and versatility is high.

The above-described embodiments are merely examples of the present invention in all respects. It goes without saying that various improvements and modifications can be made without departing from the scope of the present invention. In other words, in the implementation of the present invention, a specific configuration according to the embodiment may be adopted as appropriate.

### REFERENCE SIGNS LIST

1. Blood pressure measuring device
3. Device main body
4. Belt
5. Curler
5a. Protrusion
6. Cuff structure
7. Fluid circuit
7a. First flow path
7b. Second flow path
7c. Third flow path
11. Case
11a. Attachment portion
12. Display
13. Operation unit
14. Pump
15. Flow path section
16. On-off valve
16A. First on-off valve
16B. Second on-off valve
17. Pressure sensor
17A. First pressure sensor
17B. Second pressure sensor
18. Power supply unit
19. Vibration motor
20. Control substrate
31. Outer case
31a. Lug
31b. Spring bar
32. Windshield
33. Base
34. Flow path cover
34a. Connected portion
35. Back cover
35a. Screw
36. Flow path tube
41. Button
42. Sensor
43. Touch panel
51. Substrate
52. Acceleration sensor
53. Communication unit
54. Storage
55. Controller
61. First belt
61A. Preform
61a. First hole
61b. Second hole
61c. Buckle
61d. Frame-shaped body
61e. Prodding stick
62. Second belt
62a. Small hole
63. Cover layer
64. First insert layer
64A. First insert material
65. Second insert layer
65A. Second insert material
71. Pressing cuff
72. Back plate
72a. Groove
73. Sensing cuff
74. Bag-shaped cover body
81. Bag-shaped structure
81. Air bag
82. Tube
83. Connection portion
86. Sheet member
86a. First sheet member
86a1. Outer surface
86b. Second sheet member
86b1. Opening
86c. Third sheet member
86c1. Opening
86d. Fourth sheet member
91. Bag-shaped structure
91. Air bag
92. Tube
93. Connection portion
96. Sheet member
96a. Fifth sheet member
96b. Sixth sheet member
100. Wrist
110. Artery

1. A belt comprising:
a first belt portion including a cover layer formed of a resin material and a first insert layer disposed within the cover layer and formed of a high-tensile material having a tensile strength higher than that of the resin material, the first belt portion having a bent shape along a circumferential direction of a living body; and
a second belt portion configured to be connected to the first belt portion.

2. The belt according to claim 1, wherein:
the first belt portion further includes a second insert layer disposed within the cover layer and formed of a thermoplastic resin;
the cover layer is formed of a thermosetting resin; and
a tensile strength of the high-tensile material in the circumferential direction of the living body is higher than that of the thermosetting resin forming the cover layer.

3. The belt according to claim 1, wherein the high-tensile material includes at least one of a high-strength polyarylate fiber, a liquid crystal polymer, a PET resin, or a PEN resin.

4. The belt according to claim 1, wherein the high-tensile material is formed in a mesh shape.

5. A blood pressure measuring device comprising:
the belt according to claim 1;
a curler disposed on a side of the belt closer to a living body and having a bent shape along the living body;
a bag-shaped cuff disposed on one side of the curler, configured to be wound around the living body, and further configured to be inflated when a fluid is supplied to an inner space of the cuff; and
a supply device configured to be attached to the belt, also configured to form a flow path connected to the inner space of the cuff, and further configured to supply the fluid to the cuff.

6. A method of manufacturing a belt, the method comprising:
a preform forming step of forming a band-shaped preform by insert molding, the preform comprising a first insert material disposed inside a cover layer formed of a thermosetting resin, the first insert material made of a high-tensile material having a tensile strength higher than that of the thermosetting resin; and
a bending step of bending the preform.

7. The method of manufacturing a belt according to claim 6, wherein
the preform forming step includes: disposing a thermosetting resin material forming the cover layer around the first insert material and a second insert material that is made of a thermoplastic resin; curing the thermosetting resin and softening the second insert material by heating; thereby performing insert molding of the preform, and
the bending step includes a curing step of bending the preform and curing the second insert material at a temperature lower than a temperature at which the insert molding is performed.
